(19) United States
(12) Patent Application Publication
(10) Pub. No.: US 2022/0192963 A1
(43) Pub. Date: Jun. 23, 2022

(54) METHODS OF CURING DICARBONYL SUBSTITUTED-1-ALKENES

(71) Applicant: Sirrus, Inc., Loveland, OH (US)

(72) Inventors: Alexander Y. Polykarpov, Brunswick, OH (US); J. Taylor Goodrich, Loveland, OH (US); Anushree Deshpande, Cincinnati, OH (US)

(21) Appl. No.: 17/405,566

(22) Filed: Aug. 18, 2021

Related U.S. Application Data
(60) Provisional application No. 63/128,946, filed on Dec. 22, 2020.

(51) Int. Cl. A61K 8/81; A61Q 3/02; C08F 222/14; C09D 4/00

(52) U.S. Cl. CPC A61K 8/8135; A61Q 3/02; A61K 2800/95; C09D 4/00; C08F 222/14

(57) ABSTRACT

An article may be formed by contacting a 1,1-dicarbonyl substituted alkene with an activator comprised of an initiating salt and a weak acid and allowing the 1,1-dicarbonyl substituted alkene to cure to form the article. The article may be any useful shape such as a film, coating, layer (e.g., within an additive manufactured article), laminate or the like. The article may be one in which the cured article adheres substrates together.

### FIELD

The invention relates to improved methods for activating the polymerization or cure of 1,1-dicarbonyl substituted alkenes and in particular curing of coatings or layers thereof.

### BACKGROUND

Previously, the surface initiated cure of 1,1-dicarbonyl substituted alkenes has been achieved by coating a substrate with a base or an initiating salt followed by the application of the 1,1-dicarbonyl substituted alkenes to the coated activated substrate as described in U.S. Pat. No. 9,181,365. The coating or layers activated thereby were reported to cure after a couple or several hours.

Consequently, there is a need for a method and compositions that allow for the rapid and complete cure of coatings, films, layers, laminates and the like in applications such as nail polishes, adhesives, additive manufactured articles and wire or fiber and optic fiber coatings and adhesives.

### SUMMARY

Surprisingly films, coatings and the like of 1,1-dicarbonyl substituted alkenes (“monomer”) such as malonates cure significantly faster when they are contacted with an activator comprised of a salt and a weak acid(“activator”) compared to contacting such monomers with only the salt activator. It is unclear why this is so, but without being limited in any way, it is believed that the weak acid may initially slow the addition polymerization of the monomer at the contact with the activator allowing for improved diffusion into the monomer film of the activator allowing for substantially overall cure of the thickness of the film.

The first aspect of the invention is a method of forming an article comprised of contacting a 1,1-dicarbonyl substituted alkene with an activator comprised of an initiating salt and a weak acid having a pKa of about 3 to 14 and allowing the 1,1-dicarbonyl substituted alkene to cure to form the article. The activator may be contacted by any suitable method, but in an embodiment the activator is a liquid (i.e., does not crystallize at the contacting temperature), with a particular embodiment wherein the activator is a liquid at ambient conditions (e.g., 20 to 25° C.).

A second aspect of the invention is a film comprised of a polymerized 1,1-dicarbonyl substituted alkene, the film having a thickness from a first surface to the opposing surface of the film, wherein the film has a characteristic that varies from the first surface to the opposing surface across the thickness.

A third aspect of the invention is an article comprised a polymerized 1,1-dicarbonyl substituted alkene, the article having an outer surface and an interior bulk wherein the properties at the surface are at least 10% different by one or more characteristics.

A fourth aspect of the invention is an activation system comprised of a substrate at least partially coated with an activator comprised of an initiating salt and a weak acid.

The properties of the article may vary widely depending on the monomer and the amount and type of weak acid used allowing for articles that may vary from elastic to rigid as well as have a wide range of glass transition temperatures (Tg). The article may also have varying properties from the surface to the interior bulk or to an opposing surface such as film or coating. As such, the curable compositions and articles made therefrom may be suitable for a myriad of applications such as coatings, adhesives, additive manufactured articles, molding resins, various printing inks including inkjet inks, dental applications (e.g., fillings, inserts and the like) and medical applications among others.

### DETAILED DESCRIPTION

The explanations and illustrations presented herein are intended to acquaint others skilled in the art with the invention, its principles, and its practical application. The specific embodiments of the present disclosure as set forth are not intended to be exhaustive or limit the scope of the disclosure.

One or more as used herein means that at least one, or more than one, of the recited components may be used as disclosed. Nominal as used with respect to functionality means the theoretical functionality, generally this can be calculated from the stoichiometry of the ingredients used. Generally, the actual functionality is different due to imperfections in raw materials, incomplete conversion of the reactants and formation of by-products. Residual content of a component refers to the amount of the component present in free form or reacted with another material, such as an oligomer or a polymer. Typically, the residual content of a component can be calculated from the ingredients utilized to prepare the component or composition. Alternatively, it can be determined utilizing known analytical techniques.

### The 1,1-dicarbonyl alkene (“Monomer”)

The method to make an article of the invention uses a 1,1-dicarbonyl alkene that is contacted with an activator. The 1,1-dicarbonyl alkene compounds are for convenience referred to as “1,1-dicarbonyl alkene monomers” or just “monomer” interchangeably. The 1,1-dicarbonyl alkene monomers are compounds wherein a central carbon atom is doubly bonded to another carbon atom to form an ethylene group. The central carbon atom is further bonded to two carbonyl groups. Each carbonyl group is bonded to a hydrocarbyl group through a direct bond or an oxygen atom. Where the hydrocarbyl group is bonded to the carbonyl group through a direct bond, a keto group is formed. Where the hydrocarbyl group is bonded to the carbonyl group through an oxygen atom, an ester group is formed. The 1,1-dicarbonyl alkene may have a structure as shown below in Formula I, where X¹ and X² are an oxygen atom or a direct bond (preferably an oxygen), and where R¹ and R² are each hydrocarbyl groups that may be the same or different. Both X¹ and X² may be oxygen atoms, such as illustrated in Formula IIA, one of X¹ and X² may be an oxygen atom and the other may be a direct bond, such as shown in Formula IIB, or both X¹ and X² are direct bonds, such as illustrated in Formula IIC. The 1,1-dicarbonyl alkene compounds used herein may have all ester groups (such as illustrated in Formula IIA), all keto groups (such as illustrated in Formula IIC) or a mixture thereof (such as illustrated in Formula IIB). Compounds with all ester groups may be preferred in some applications due to the flexibility of synthesizing a variety of such compounds.

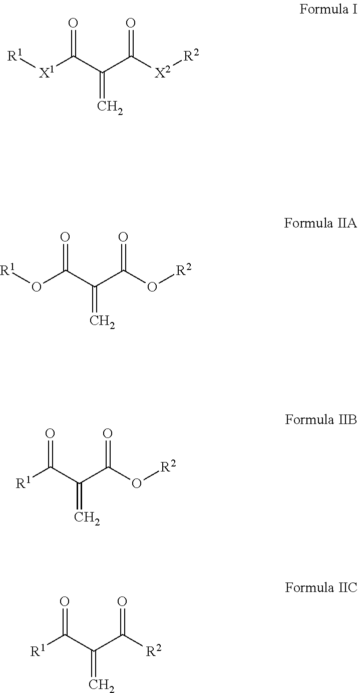

Heteroatom means nitrogen, oxygen, sulfur and phosphorus, more preferred heteroatoms include nitrogen and oxygen. Hydrocarbyl as used herein refers to a group containing one or more carbon atom backbones and hydrogen atoms, which may optionally contain one or more heteroatoms. Where the hydrocarbyl group contains heteroatoms, the heteroatoms may form one or more functional groups well known to one skilled in the art. Hydrocarbyl groups may contain cycloaliphatic, aliphatic, aromatic or any combination of such segments. The aliphatic segments can be straight or branched. The aliphatic and cycloaliphatic segments may include one or more double and/or triple bonds. Included in hydrocarbyl groups are alkyl, alkenyl, alkynyl, aryl, cycloalkyl, cycloalkenyl, alkaryl and aralkyl groups. Cycloaliphatic groups may contain both cyclic portions and noncyclic portions. Hydrocarbylene means a hydrocarbyl group or any of the described subsets having more than one valence, such as alkylene, alkenylene, alkynylene, arylene, cycloalkylene, cycloalkenylene, alkarylene and aralkylene. One or both hydrocarbyl groups may consist of one or more carbon atoms and one or more hydrogen atoms. As used herein percent by weight or parts by weight refer to, or are based on, the weight of the solution composition unless otherwise specified.

The 1,1-dicarbonyl alkene compounds may be methylene malonates which refer to compounds as shown below:

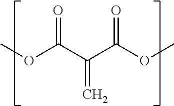

The term “monofunctional” refers to 1,1-dicarbonyl alkene compounds or a methylene malonate having only one core formula. The term “difunctional” refers to 1,1-dicarbonyl alkene compounds or a methylene malonate having two core formulas bound through a hydrocarbyl linkage between one oxygen atom on each of two core formulas. The term “multifunctional” refers to 1,1-dicarbonyl alkene compounds or methylene malonates having more than one core formula which forms a chain through a hydrocarbyl linkage between one oxygen atom on each of two adjacent core formulas.

The 1,1-dicarbonyl alkene monomer may be a 1,1-diester-1-alkene. As used herein, diester refers to any compound having two ester groups. A 1,1-diester-1-alkene is a compound that contains two ester groups and a double bond bonded to a single carbon atom referred to as the one carbon atom. Dihydrocarbyl dicarboxylates are diesters having a hydrocarbylene group between the ester groups wherein a double bond is not bonded to a carbon atom which is bonded to two carbonyl groups of the diester.

The hydrocarbyl groups (e.g., Ft′ and R²), each may comprise straight or branched chain alkyl, straight or branched chain alkyl alkenyl, straight or branched chain alkynyl, cycloalkyl, alkyl substituted cycloalkyl, aryl, aralkyl, or alkaryl. The hydrocarbyl group may optionally include one or more heteroatoms in the backbone of the hydrocarbyl group. The hydrocarbyl group may be substituted with a substituent that does not negatively impact the ultimate function of the monomer or the polymer prepared from the monomer. Preferred substituents include alkyl, halo, alkoxy, alkylthio, hydroxyl, nitro, cyano, azido, carboxy, acyloxy, and sulfonyl groups. More preferred substituents include alkyl, halogen, alkoxy, allylthio, and hydroxyl groups. Most preferred substituents include halogen, alkyl, and alkoxy groups.

As used herein, alkaryl means an alkyl group with an aryl group bonded thereto. As used herein, aralkyl means an aryl group with an alkyl group bonded thereto and include alkylene bridged aryl groups such as diphenyl methyl groups or diphenyl propyl groups. As used herein, an aryl group may include one or more aromatic rings. Cycloalkyl groups include groups containing one or more rings, optionally including bridged rings. As used herein, alkyl substituted cycloalkyl means a cycloalkyl group having one or more alkyl groups bonded to the cycloalkyl ring.

The hydrocarbyl groups may include 1 to 30 carbon atoms, 1 to 20 carbon atoms, or 1 to 12 carbon atoms. Hydrocarbyl groups with heteroatoms in the backbone may be alkyl ethers having one or more alkyl ether groups or one or more alkylene oxy groups. Alkyl ether groups may be ethoxy, propoxy, and butoxy. Such compounds may contain from about 1 to about 100 alkylene oxy groups, about 1 to about 40 alkylene oxy groups, about 1 to about 12 alkylene oxy groups, or about 1 to about 6 alkylene oxy groups.

One or more of the hydrocarbyl groups (e.g., R¹, R2, or both) may include a C1-15 straight or branched chain alkyl, a C1-C15 straight or branched chain alkenyl, a C5-C18 cycloalkyl, a C6-C24 alkyl substituted cycloalkyl, a C4-C18 aryl, a C4-C20 aralkyl, or a C4-C20 aralkyl. The hydrocarbyl group may include a C1-C8 straight or branched chain alkyl, a C5-C12 cycloalkyl, a C6-C12 alkyl substituted cycloalkyl, a C4-C18 aryl, a C4-C20 aralkyl, or a C4-C20 aralkyl.

Alkyl groups may include methyl, propyl, isopropyl, butyl, tertiary butyl, hexyl, ethyl pentyl, and hexyl groups. More preferred alkyl groups include methyl and ethyl. Cycloalkyl groups may include cyclohexyl and fenchyl. Alkyl substituted groups may include menthyl and isobornyl, norbornyl as well as any other bicyclic, tricyclic or polycyclic structure.

Hydrocarbyl groups attached to the carbonyl group may include methyl, ethyl, propyl, isopropyl, butyl, tertiary, pentyl, hexyl, octyl, fenchyl, menthyl, and isobornyl, cyclic, bicyclic or a tricyclic group such as cyclohexyl, norbornyl, or tricyclodecanyl.

Monomers may include methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

Some or all of the 1,1-dicarbonyl alkene can also be multifunctional, having more than one core unit and thus more than one alkene group. Exemplary multifunctional 1,1-dicarbonyl alkenes are illustrated by the formula:

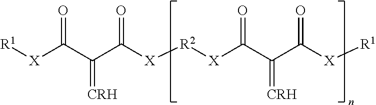

wherein R¹ and R² are as previously defined; X is, separately in each occurrence, an oxygen atom or a direct bond; n is an integer of 1 or greater to any useful amount such as a polymer of 1,000 or 10,000 Daltons or more to typically at most about 1,000,000 or 100,000 and R is hydrogen or a hydrocarbyl group having 1 to 30 carbons, so long as at least one R is hydrogen (i.e., ═CH₂) and preferably every R is hydrogen. Typically, n is 1 or 2 to 20 or 10. In another embodiment, the monomer may be an oligomer or polymer as depicted above, but the methylene malonate is replaced with a malonate or di-carbonyl lacking the ═CH₂ between the carbonyls so long as one of the repeating units has the methylene group (═CH₂) between the carbonyls.

In exemplary embodiments R² may be, separately in each occurrence, straight or branched chain alkyl, straight or branched chain alkenyl, straight or branched chain alkynyl, cycloalkyl, alkyl substituted cycloalkyl, aryl, aralkyl, or alkaryl, wherein the hydrocarbyl groups may contain one or more heteroatoms in the backbone of the hydrocarbyl group and may be substituted with a substituent that does not negatively impact the ultimate function of the compounds or polymers prepared from the compounds. Exemplary substituents may be those disclosed as useful with respect to R1. In certain embodiments R² may be, separately in each occurrence, C₁₋₁₅ straight or branched chain alkyl, C₂₋₁₅ straight or branched chain alkenyl, C₅₋₁₈ cycloalkyl, C₆₋₂₄ alkyl substituted cycloalkyl, C₄₋₁₈ aryl, C₄₋₂₀ aralkyl or C₄₋₂₀ aralkyl groups. In certain embodiments R² may be separately in each occurrence C 1-8 straight or branched chain alkyl, C₅₋₁₂ cycloalkyl, C₆₋₁₂ alkyl substituted cycloalkyl, C₄₋₁₈ aryl, C₄₋₂₀ aralkyl or C₄₋₂₀ alkaryl groups.

In an embodiment, X is O and R² is the residue of a diol, wherein a polyester is formed. The polyesters may be formed from any suitable 1,1-dicarbonyl alkene such as the malonates described above and as described in U.S. Pat. No. 9,969,822 from col. 19, line 49 to col. 20, line 3 and a polyol incorporated herein by reference. Examples of suitable polyol include, for example, those described in U.S. Pat. No. 9,969,822 from col. 20, line 18 to col. 21, line 26, incorporated herein by reference. Examples of diols may include ethylene diol 1,3-propylene diol, 1,2 propylene diol, 1-4-butanediol, 1,2-butane diol, 1,3-butane diol, 2,3-butane diol, 1,5-pentane diol, 1,3- and 1,4-cyclohexanedimethanols or combinations thereof. Examples of triols may include, glycerol, 1,2,3-propane triol, 1,2,3-butane triol, trimethylolpropane, 1,2,4-butane triol or combination thereof. Likewise, the polyol may be even higher functional, for example, di(trimethylolpropane), pentaerythritol, dipentaerythritol or combination thereof. Any combination of polyols such as multiple diols, triols, tetraols, pentaols, hexaols or mixtures thereof may be used.

The 1,1-dicarbonyl alkene monomers may be produced and purified by the methods described in U.S. Pat. Nos. 8,609,8985; 8,884,051; 9,108,914 and 9,518,001 and Int. Pub. WO 2017/197212. Examples of such monomers are available under the tradenames CHEMILIAN and FORZA and include, for example, methylene malonate, dihexyl methylene malonate, dicyclohexyl methylene malonate and multifunctional polyester methylene malonates available from Sirrus, Inc., Loveland, Ohio.

In an embodiment, the monomer may also contain a weak acid. The weak acid is the same as described below. Typically, the amount of weak acid in the monomer is at most about 10%, 5% or 2% by weight of the monomer and weak acid. Desirably, the weak acid present in the monomer is a carboxylic acid such as those described below. In another embodiment, the 1,1-dcicarbonyl alkene monomers may be comprised of monomers having other chemistries that may copolymerize with the 1,1-dicarbonyl alkene monomers such as those that may undergo anionic or radical polymerization through an unsaturated bond. Typically, when such other monomers are present they are present in minor amount such as less than 50% or 10% by weight to about 0.1% or 1% by weight of the total amount of monomers including the other monomers. An example of such other monomers is cyanoacrylates.

### Activator

The method contacts the monomer with an activator. The activator is comprised of an initiating salt and weak acid as described below. It is desirable for the activator to be a liquid at the temperature where the monomer is contacted with the activator as well as the time it takes to cure the monomer. The contacting temperature may be any useful temperature. For example, a substrate having the activator on a surface thereof may be heated above the melting temperature of the activator and then the monomer is coated there on by any suitable method such as those known in the art (e.g., spraying, painting, rolling or the like). Desirably, the melting point of the activator is less than 100° C., 75° C., 50° C. or 30° C. or is a liquid at ambient conditions/temperatures (20° C. to 25° C.). In an embodiment, the activator, for example, may be coated while a liquid or using a solvent where the activator layer is a solid at ambient temperatures, the monomer is then coated upon the solid activator layer and then heated to a temperature where the activator is a liquid thereby polymerizing the monomer.

The activator may be provided in any useful amount to cause the activation of the monomer. Illustratively the activator is coated on a substrate at any useful thickness and the coating uniformity may be aided by use of a solvent, which may be removed by evaporation aided or otherwise (e.g., application of heat or vacuum). In an embodiment the activator may be place on two or more substrates and the monomer contacted with both such that the monomer is interposed between the substrates adhering them together. In another embodiment, the activator may be contacted with sequentially deposited layers of monomer and activator at particular locations of each monomer layer so as to form additive manufactured articles.

The thickness of the activating layers may be any useful for a particular application. Typically, the thickness of the activating layer is from about 1 micrometers (μm), 2 μm, 10 μm, 25 μm, 50 μm, 75 μm or 100 μm to several millimeters (mm), 2 mm, 1 mm or 0.5 mm. Illustratively, but not limiting in any way, the activating layer thickness to monomer layer thickness ratio (activating layer thickness/monomer layer thickness) forming a film or coating may vary from about 0.01 0.05, 0.1, 0.2, 0.5 to 2, 1.5 or about 1.

The amount of initiating salt and weak acid present in the activator may be any useful amount depending on the ultimately desired properties in the article formed. For example, higher ratios (molar or equivalence ratios) of initiating salt/weak acid tends to form articles having higher hardness or stiffness (higher elastic modulus) whereas when the ratios are lower the article tends to be softer or more elastic (e.g., lower elastic modulus). Generally, the initiating salt/weak acid molar or equivalence ratio may be from about 0.1, 0.2, 0.5, 1 to 200, 100, 50, 25, 20, 15, 10 or 5.

In an embodiment, the salt is comprised of an anion that is the same as the anion of the weak acid forming the salt. For example, the salt is of an amine (e.g., mono or multifunctional amine) and monocarboxylic acid with the monocarboxylic acid being provided in a molar excess of the stoichiometric amount needed to form the salt. The weak acid may also be different than the weak acid used to form the salt or any combination of the weak acids described herein, including those used to make the salt. The excess amount in such embodiment may be as described above. Salts of multifunctional amines with multifunctional acids may also be used.

### Initiating Salt

The initiating salt may be any useful salt that leads to one or more of the desirable characteristics described for the activator (e.g., liquid at room temperature). It is understood that the salt is of an equivalent stoichiometric mixture of a base and the weak acid. Illustratively, the initiating salt may be comprised of one or more of sodium acetate; potassium acetate; acid salts of sodium, potassium, lithium, copper, and cobalt; tetrabutyl ammonium fluoride, and chloride; benzoate salts; 2,4-pentanedionate salts; sorbate salts; propionate salts; salts of amines with monocarboxylic acids; piperidine acetate; metal salts of a carboxylic acid; copper(II) acetate, cupric acetate monohydrate, potassium acetate, zinc acetate, zinc chloracetate, magnesium chloracetate, magnesium acetate; salts of acid containing polymers; or salts of polyacrylic acid co-polymers. N,N-dimethylbenzylamine octanoate, N,N-dimethylbenzylamine neodecanoate.

In an embodiment the initiating salt is comprised of an amine and a carboxylic acid. The carboxylic acid may be any one of those described herein for the weak acid. Desirably, the amine and carboxylic acid are monofunctional.

A solvent may be used to dissolve the initiating salt or any portion of the activator, for example, to aid in the application to a substrate to form an activator layer described herein. The solvent may be any solvent that dissolves the salt and/or weak acid. Generally, any polar solvent such as alcohols, ketones, ethers, esters, a protic solvent or the like may be used. In an embodiment, a carrier polymer may be used to dissolve or disperse a portion or all of the activator (salt and/or weak acid).

### Weak Acid

The weak acid may be any compound that is a weak acid displaying a pKa of about 3, 3.5, 4 to 14, 12, 10, 8, 6, 5.5 or 5 with it being understood that pKa is the logarithm of the acid disassociation constant in water. It is also understood that in some cases where multiple carboxylic acid groups are contained in a polymer, the pKa may be difficult to ascertain, but these are contemplated herein. The weak acid may be multifunctional (i.e., 2 or more acids), but desirably is monofunctional. The weak acid may be saturated or unsaturated. The weak acid may be any carboxylic acid such as a natural occurring carboxylic acid. For example, the carboxylic acid may be abietic acid, alginic acid, gallic acid, azelaic acid, caffeic acid, malic acid, pyruvic acid, niacin, citric acid, biotin, abietic acid, cholic resin, pectin, alginic acid, gum rosin (a mixture of naturally occurring natural acids) or combination thereof. The fatty acid may be any fatty acid derived from any animal fat or vegetable oil and maybe saturated or unsaturated. Exemplary oils include linseed, palm, coconut, palm, olive, tung, soybean, peanut, sunflower, cotton seed, rapeseed, or combination thereof. Any fatty acid derived from the aforementioned oils and fats may be used (e.g., isostearic acid). In an embodiment, the fatty acid is dimerized to form a dimer, trimer acid or higher polymeric acids. Typically, readily available dimer acids contain some small fraction of monomeric acid, trimer acid and higher polymeric acid. Such Dimer acids are readily available, for example, from Oleon NV. Ertevelde, Belgium under the tradename RADIACID.

The weak acid may be any carboxylic acid such as mono or dicarboxylic acids having linear or branched alkane chains of 3 or 5 to 30, 20 or 15 carbons or mixtures thereof. Examples may include hexanoic, hexanedioic acid, heptanoic acid, heptanedioic acid, octanoic acid, octanedioic acid, nonanoic acid, nonanedioic acid, decanoic acid, decanedioic acid, 2,3-dimethylbutaneoic acid, 2,3-dimethylbutaneoic acid, 2,3-dimethylbutanedioic, 2,2-dimethylbutaneoic acid, 2,2-dimethylbutanedioic acid, 3-methylheptaneoic acid, 3-methylheptanedioic acid or mixtures thereof.

The weak acid may be a sterically hindered carboxylic acid with a short and highly branched alkyl chemical structure such as 2,2,3,5-Tetramethylhexanoic acid, 2,4-Dimethyl-2-isopropylpentanoic acid, 2,5-Dimethyl-2-ethylhexanoic acid, 2,2-Dimethyloctanoic acid, 2,2-Diethylhexanoic acid or those available under the tradename VERSATIC acids from Hexion Inc. Columbus, Ohio.

The weak acid may also be a carboxyl containing: polyether, polyester, polyether-ester, polyamide, conjugated diene polymer or conjugated diene copolymer, polyurethane, polystyrene, polyolefin, silicone or combination thereof. The weak acid may also be a polysaccharide, polypeptide, protein or combination thereof.

The activator or monomer may be comprised of further components that may vary depending on the application. The further components may be one or more dyes, pigments, toughening agents, impact modifiers, rheology modifiers, natural or synthetic rubbers, filler agents, reinforcing agents, thickening agents, opacifiers, inhibitors, fluorescence markers, thermal degradation reducers, thermal resistance conferring agents, surfactants, wetting agents, leveling agents or stabilizers can be included in the activator or monomer. In certain embodiments, such thickening agents and other compounds can be used to increase the viscosity the monomer or activator from about 1 to 3 centipoise (cP) to about 30,000 cP, or more.

The activator or monomer may contain a filler in certain embodiments such as printing dyes or additive manufacturing techniques such as polyjeting and inkjetting, which are described below. Examples of filler include talc, wollastonite, mica, clay, montmorillonite, smectite, kaolin, calcium carbonate, glass fibers, glass beads, glass balloons, glass milled fibers, glass flakes, carbon fibers, carbon flakes, carbon beads, carbon milled fibers, metal flakes, metal fibers, metal coated glass fibers, metal coated carbon fibers, metal coated glass flakes, silica, other ceramic particles, ceramic fibers, ceramic balloons, aramid particles, aramid fibers, polyarylate fibers, graphite, and various whiskers such as potassium titanate whiskers, aluminum borate whiskers and basic magnesium sulfate whiskers. The fillers may be incorporated alone or in combination.

The method may be used in any number of applications. Exemplary applications include films, additive manufactured articles, nail polish, caulks, adhesives, sealants, coatings (e.g., metal panels, glass, fibers, fabrics, wood or composites coatings and adhesives). Illustratively the nail polish may be, but need not be, multilayered to form for example an underlying color coating and a top clear coat (e.g., sequentially an activator coat on the nail, colorant coat, monomer coat, activator coat and finally a top monomer coat). In another embodiment the method may be used to bond fibers into a ferule that then can be used to integrate the bundle into other devices.

When making an article the monomer is contacted with the activator. The contacting may occur by any useful method such as those known in the art. Illustratively, the activator may be provided as a coating on at least a portion of a substrate (i.e., “activation system” system herein). The substrate may be any substrate which may desirably coated (wires, plates, metal coils, fibers, synthetic or natural finger nails) or adhered to a separate substrate forming a laminated structure. The substrate may be a particulate with the activator thereon that may form a paste when contacted with the monomer, the paste may then be deposited on a further substrate to form a caulk, coating or adhesive on the further substrate. The contacting may occur by printing, spraying, brushing, rolling, troweling, doctor blading or the like of the activator on to a layer of monomer or vice versa such as may be encountered in additive manufacturing. The contacting may occur under any useful conditions as previously described. Desirably, the temperature is such that the activator is a liquid or amorphous in character. The contacting may occur at any contacting temperature as previously described. The contacting temperature may be held constant during the curing of the monomer.

The method and activator system allows the polymerization/cure of the monomer in short times (e.g., less than 15, 10, 5, 2 or even one minute) while varying the properties of the film or article produced thereby. For example, without be limiting in any way, an article may be formed by coating a mold surface with a mold release that is then coated with the activator where the monomer is injected or cast into the mold to form the article.

In another embodiment, the activation system is comprised of a substrate at least partially coated with the activator. The substrate may be any useful substrate such as a ceramic, metal, metalloid, glass, plastic, wood, a composite of any of the aforementioned, or combination thereof. In another embodiment, the substrate may be a synthetic fingernail or natural fingernail. Desirably, the activation systems are comprised of an activator that is liquid under ambient temperatures (e.g., both the initiating salt and weak acid are liquids).

It has been discovered that the article formed by the method of this invention allows the formation of articles that may vary in property characteristics from the monomer's first point/surface of contact with the activator to within the bulk of the article. In the case of a film or coating the property characteristic may vary from the surface first contacted with the activator to the opposing surface of a film or coating not contacted. Alternatively, if the film or layer has been contacted on both surfaces the property characteristic may vary from both surfaces into the bulk and may vary the same or differently depending on the composition of the activator contacting each surface.

Illustratively, the characteristic that changes even though the monomer is the same, is the stiffness (elastic modulus) or hardness. The property difference may vary by 10%, 20% even 50% or more to several thousand percent from the contact surface to the bulk or the other surface of a film. The properties may be determined by known techniques such as nanoindentation across the thickness of film or cross-section of an article into the bulk. It is understood the surface extends into the thickness of the film or article bulk some amount such as 1 to 5 micrometers. In an embodiment, the characteristic is a gradient without any step changes in the property.

### Illustrative Embodiments

Embodiment 1. A method of forming an article comprised of;
(ii) contacting a 1,1-dicarbonyl substituted alkene with an activator comprised of an initiating salt and a weak acid having a pKa of about 3 to 14,
(iii) allowing the 1,1-dicarbonyl substituted alkene to cure to form the article.

Embodiment 2. The method of Embodiment 1, wherein the activator salt is comprised an amine and a carboxylic acid.

Embodiment 3. The method of any one of the preceding Embodiments, wherein the weak acid is a carboxylic acid.

Embodiment 4. The method of Embodiments 2 or 3, wherein the carboxylic acid of the initiating salt is the same as the weak acid.

Embodiment 5. The method of any one of the preceding Embodiments, wherein the weak acid is present in the activator in an amount such that the equivalent ratio of initiating salt/weak acid is from 0.2 to 20.

Embodiment 6. The method of Embodiment 5, wherein the equivalent ratio of initiating salt/weak acid is from 0.2 to 10.

Embodiment 7. The method of any one of the preceding Embodiments, wherein the initiating salt is a liquid at the temperature where the 1,1-dicarbonyl substituted alkene is contacted with the activator.

Embodiment 8. The method of any one of the preceding Embodiments, wherein the initiating salt is a liquid at ambient conditions.

Embodiment 9. The method of any one of the preceding Embodiments, wherein the 1,1-dicarbonyl substituted alkene is represented by:

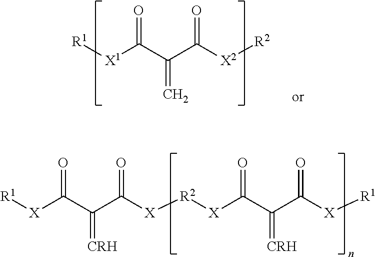

wherein X, X¹ and X² are an oxygen atom or a direct bond, and where R¹ and R² are each hydrocarbyl groups having from 1 to 30 carbons and R is hydrogen or a hydrcarbyl group having from 1 to 30 carbons, so long as at least one R is hydrogen.

Embodiment 11. The method of Embodiment 10, wherein the 1,1-dicarbonyl alkene monomer is a methylene malonate.

Embodiment 12. The method of any of the preceding Embodiments, wherein the activator is applied to a substrate prior to be contacted with the 1,1-dicarbonyl substituted alkene.

Embodiment 13. The method of any one of the preceding Embodiments, wherein the activator is dissolved in a solvent and the solvent prior to contacting with the 1,1-dicarbonyl substituted alkene is removed.

Embodiment 14. The method of any one of the preceding Embodiments, wherein the initiating salt is comprised of one or more of sodium acetate; potassium acetate; acid salts of sodium, potassium, lithium, copper, and cobalt; tetrabutyl ammonium fluoride, chloride, and hydroxide; benzoate salts; 2,4-pentanedionate salts; sorbate salts; propionate salts; salts of amines with organic monocarboxylic acids; piperidine acetate; metal salts of a monocarboxylic acid; copper(II) acetate, cupric acetate monohydrate, potassium acetate, zinc acetate, zinc chloracetate, magnesium chloracetate, magnesium acetate; salts of acid containing polymers; or salts of polyacrylic acid co-polymers.

Embodiment 15. The method of any one of the preceding Embodiments, wherein the initiating salt is comprised of a salt of an amine with a monocarboxylic acid.

Embodiment 16. The method of Embodiment 15, wherein the monocarboxylic acid is C4 to C36 linear or branched carboxylic acid.

Embodiment 17. The method of any one of the preceding Embodiments, wherein the allowing to cure of the 1,1-dicarbonyl substituted alkene is at ambient temperature and the initiating salt is a liquid.

Embodiment 18. The method of any one of the preceding Embodiments, wherein the 1,1-dicarbonyl substituted alkene is further comprised of a weak acid having a pKa of 3 to 14.

Embodiment 19. The method of Embodiment 18, wherein the weak acid is present in the 1,1-dicarbonyl substituted alkene in an amount of at most about 10% by weight of the 1,1-dicarbonyl substituted alkene and weak acid.

Embodiment 20. The method of either Embodiment 18 or 19, wherein the weak acid present in the 1,1-dicarbonyl substituted alkene is a carboxylic acid.

Embodiment 21. The method any one of the preceding Embodiments, wherein the article is a coating, film, or layer in an additive manufactured article.

Embodiment 22. An article made by the method of any one of the preceding Embodiments.

Embodiment 23. A film comprised of a polymerized 1,1-dicarbonyl substituted alkene, the film having a thickness from a first surface to the opposing surface of the film, wherein the film has a gradient characteristic from the one surface to the opposing surface across the thickness.

Embodiment 24. The film of Embodiment 23, wherein the gradient characteristic is hardness that varies by at least 10% from the one surface to the opposing surface.

Embodiment 25. The film of Embodiment 24, wherein the hardness varies by at least 20% different.

Embodiment 26. The film of any one of the preceding Embodiments 23 to 25, wherein the film is a coating or adhesive layer bonding two substrates.

Embodiment 27. An article comprised a polymerized 1,1-dicarbonyl substituted alkene, the article having an outer surface and an interior bulk wherein the properties at the surface are at least 10% different by one or more characteristics.

Embodiment 28. An activation system comprised of a substrate at least partially coated with an activator comprised of an initiating salt and a weak acid.

Embodiment 29. The activation system of Embodiment 28, wherein the substrate is a ceramic, metal, metalloid, glass, plastic, wood, a composite of any of the aforementioned, or combination thereof.

Embodiment 30. The activation system of Embodiment 28, wherein the substrate is a fingernail or synthetic fingernail.

Embodiment 31. The activator system of any one of Embodiments 28 to 30, wherein the activator is a liquid at ambient temperature.

Embodiment 32. The method of any one of Embodiments 1 to 21, wherein the weak acid is a naturally occurring carboxylic acid, dimerized fatty acid, a sterically hindered carboxylic acid with a short and highly branched alkyl chemical structure, a carboxyl containing: polyether, polyester, polyether-ester, polyamide, conjugated diene polymer or conjugated diene copolymer, polyurethane, polystyrene, polyolefin, silicone or combination thereof.

### Examples

The following examples are provided to illustrate the curable compositions and the copolymers formed from them, but are not intended to limit the scope thereof. All parts and percentages are by weight unless otherwise noted. Table 1 shows the ingredients used in the Examples and Comparative Examples.

TABLE 1

| | Acronyms | | |
|---|---|---|---|
| Composition | used | | |
| component | herein | Company | Description |
| methylene | BDPES | Sirrus Inc. | Monomer: obtained from |
| malonate | | | transesterification of 1,4- |
| polyester | | | butanediol and diethyl |
| | | | methylene malonate |
| N,N-dimethyl- | DMBA | Sigma | Amine (salt cation) |
| benzylamine | | Aldrich | |
| VERSATIC 10 Acid | VERSATIC | Hexion | Sterically hindered |
| | | | carboxylic weak acid |
| | | | (neodecanoic acid) and |
| | | | (salt anion) |
| BYK-361 N | Not | BYK | Polyacrylate based |
| | Applicable | | leveling agent |
| Octanoic acid | OCT A | Sigma | Carboxylic acide |
| | | Aldrich | |
| Ethyl Cyanoacrylate | ETCYAN | WHO | Other Monomer |

### Examples and Comparative Examples

In each of the Examples the activator is prepared by mixing, for about 1 hour, the DMBA and Octanoic acid at an equivalence/molar stoichiometric ratio. In a 250 ml Erlenmeyer flask equipped with magnetic stirring was charged with an amount of Octanoic acid. To the acid N,N-dimethylbenzylamine was slowly poured into the acid to achieve the stoichiometric ratio while stirring the acid. A slight exotherm was observed and the reaction mixture became more viscous. The mixture was stirred for about 1 hour at room temperature followed by heating at 50 C for 1 hour. Excess Octanoic acid or VERSATIC acid as well as 0.3% by weight BYK leveling agent was added to the equivalence ratio of and added acid as shown in the parenthesis of Table 2.

The activator is applied to the substrate as noted in Table 2. The BDPES monomer containing the noted amount of VERSATIC acid (weight %) and Ethyl Cyanoacrylate as well as 0.3% by weight of BYK-361 leveling agent was then contacted with the activator coated substrate. The coating tack free time was measured as the time elapsed between application of the monomer layer on top of the activator and the point when dragging a 0.08″ diameter wooden dowel across the coating surface (with about 1 lb of force) no longer left a visible mark. The results are shown in Tables 2.

As can be seen from Table 2, Examples that contain excess of acid in the activator (salt) cure faster than the Comparative Examples that have no excess acid in the activator. The difference in curing speed is even more pronounced for thicker coatings. The Examples with the DMBA salt in the activating layer cured faster than the Comparative Example using a DMBA free amine activating layer. Examples containing a blend of cyanoacrylate with methylene malonate also cured faster using an acidified salt activator layer.

TABLE 2

| | | Activator | Tack | Activator | Monomer | |
|---|---|---|---|---|---|---|
| | | Composition^{#} | Free | Layer | Layer | |
| Example/ | Monomer | (Salt/Excess | Time | Application | Application | |
| C. Exam. | Composition* | Acid) | (min) | Method | Method | Substrate |
| Comp. 1 | 5% VERSATIC | No excess acid | 0.62 | A | B | polycarbonate |
| 1 | 5% VERSATIC | (11.7 VERSATIC) | 0.48 | A | B | polycarbonate |
| 2 | 5% VERSATIC | (9.8 OCT A) | 0.51 | A | B | polycarbonate |
| Comp. 2 | 5% VERSATIC | No excess acid | 3.5 | A | B | polycarbonate |
| | 9.5% ETCYAN | | | | | |
| 3 | 5% VERSATIC | (11.7 VERSATIC) | 3 | A | B | polycarbonate |
| | 9.5% ETCYAN | | | | | |
| Comp. 3 | | Neat DMBA | 0.46 | A | B | polycarbonate |
| Comp. 4 | | No excess acid | 0.24 | A | B | polycarbonate |
| 4 | | (11.7 VERSATIC) | 0.2 | A | B | polycarbonate |
| Comp. 5 | | No excess acid | 9.5-10.5 | A | C | polycarbonate |
| 5 | | (9.8 OCT A) | 6-7 | A | C | polycarbonate |

*Balance BPDES monomer by weight and 0.3% by weight BYK leveling agent.
^{#}Salt-DMBA: Octanoic acid and excess free acid added as noted with equivalence as noted in ( ).
A: The activator was drawn down with a #2.5 Mayer coating rod
B: Monomer coating was applied with a 5-mil (~0.127 mm) square applicator (gap is 5 mil corresponding to thickness of coating)
C: Monomer coating was applied with a 10-mil (~0.254 mm) square applicator (gap is 10 mil corresponding to thickness of coating)

What is claimed is:

1. A method of forming an article comprised of;
(i) contacting a 1,1-dicarbonyl substituted alkene with an activator comprised of an initiating salt and a weak acid having a pKa of about 3 to 14,
(ii) allowing the 1,1-dicarbonyl substituted alkene to cure to form the article.

2. The method of claim 1, wherein the activator salt is comprised an amine and a carboxylic acid.

3. The method of claim 1, wherein the weak acid is a carboxylic acid.

4. The method of claim 3, wherein the carboxylic acid of the initiating salt is the same as the weak acid.

5. The method of claim 1, wherein the weak acid is present in the activator in an amount such that the equivalent ratio of initiating salt/weak acid is from 0.2 to 20.

6. The method of claim 1, wherein the initiating salt is a liquid at the temperature where the 1,1-dicarbonyl substituted alkene is contacted with the activator.

7. The method of claim 1, wherein the initiating salt is a liquid at ambient conditions.

8. The method of claim 1, wherein the 1,1-dicarbonyl substituted alkene is represented by: 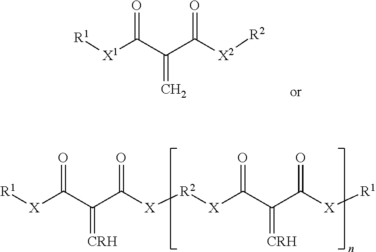
wherein X, X¹ and X² are an oxygen atom or a direct bond, and where R¹ and R² are each hydrocarbyl groups having from 1 to 30 carbons and R is hydrogen or a hydrcarbyl group having from 1 to 30 carbons, so long as at least one R is hydrogen.

9. The method of claim 8, wherein the 1,1-dicarbonyl alkene monomer is a methylene malonate.

10. The method of claim 1, wherein the activator is applied to a substrate prior to be contacted with the 1,1-dicarbonyl substituted alkene.

11. The method of claim 1, wherein the activator is dissolved in a solvent and the solvent prior to contacting with the 1,1-dicarbonyl substituted alkene is removed.

12. The method of claim 1, wherein the initiating salt is comprised of a salt of an amine with a monocarboxylic acid.

13. The method of claim 1, wherein the allowing to cure of the 1,1-dicarbonyl substituted alkene is at ambient temperature and the initiating salt is a liquid.

14. The method of claim 1, wherein the 1,1-dicarbonyl substituted alkene is further comprised of a weak acid having a pKa of 3 to 14.

15. The method of claim 14, wherein the weak acid is present in the 1,1-dicarbonyl substituted alkene in an amount of at most about 10% by weight of the 1,1-dicarbonyl substituted alkene and weak acid.

16. The method of claim 1, wherein the weak acid is a naturally occurring carboxylic acid, dimerized fatty acid, a sterically hindered carboxylic acid with a short and highly branched alkyl chemical structure, a carboxyl containing: polyether, polyester, polyether-ester, polyamide, conjugated diene polymer or conjugated diene copolymer, polyurethane, polystyrene, polyolefin, silicone or combination thereof.

17. A film comprised of a polymerized 1,1-dicarbonyl substituted alkene, the film having a thickness from a first surface to the opposing surface of the film, wherein the film has a gradient characteristic from the one surface to the opposing surface across the thickness.

18. An activation system comprised of a substrate at least partially coated with an activator comprised of an initiating salt and a weak acid.

19. The activation system of claim 18, wherein the substrate is a fingernail or synthetic fingernail.

20. The activation system of claim 18, wherein the activator is a liquid at ambient temperature.
